(19) United States
(12) Patent Application Publication
(10) Pub. No.: US 2006/0241183 A1
(43) Pub. Date: Oct. 26, 2006

(54) Compositions and methods of using D-DOPA to treat Parkinson's disease

(76) Inventor: Farouk Karoum, Alexandria, VA (US)

Correspondence Address: Pepper Hamilton LLP;One Mellon Center, 50th Floor, 500 Grant Street, Pittsburgh, PA, 15219, US

(21) Appl. No.: 11/238,669

(22) Filed: Sep. 28, 2005

Related U.S. Application Data
(60) Provisional application No. 60/613,823, filed on Sep. 28, 2004.

(51) Int. Cl. A61K 31/198; A61K 31/137; A61K 31/045

(52) U.S. Cl. 514/567; 514/649; 514/727

(57) ABSTRACT

A method of treating Parkinson's disease by administering the racemic mixture of D,L-DOPA in combination with both peripheral amino acid decarboxylase and catechol, O-methyltransferase (COMT) inhibitors in pharmaceutically acceptable salts forms and effective doses for the treatment of Parkinson's disease. Alternatively, D-DOPA is administered in combination with both peripheral amino acid decarboxylase and catechol, O-methyltransferase (COMT) inhibitors in pharmaceutically acceptable salts forms and effective doses for the treatment of Parkinson's disease.

### CROSS REFERENCE TO RELATED APPLICATIONS

This application claims priority from U.S. Provisional Application No. 60/613,823 filed Sep. 28, 2004 entitled “D,L-DOPA, or D-DOPA methods of treating parkinson's disease.”

### BACKGROUND INFORMATION

Parkinson's disease is believed to be a result of cholinergic over activity due to a major reduction in dopamine synthesizing cells in the basal ganglia and the dopaminergic nerve terminals in the corpus striatum (major motor centers in the brain). Administration of dopamine, alone, is not an effective therapy since dopamine does not cross the blood brain barrier. L-DOPA, on the other hand, does cross the blood brain barrier where it is converted to dopamine in the basal ganglia. However, only a small percentage of orally administered L-DOPA actually crosses the blood brain barrier due to the peripheral conversion of L-DOPA to dopamine by L-amino acid decarboxylase and O-methylation. Further, administration of L-DOPA is almost always associated with untoward side effects such as nausea, vomiting, hypotension, abnormal movements, and behavioral changes. In order for sufficient amounts of L-DOPA to cross the blood brain barrier, large doses of L-DOPA are required. Large doses of L-DOPA exacerbate these untoward side effects. In an attempt to overcome these problems, carbidopa alone or with a catetchol-O-methyltransferase (COMT) inhibitor were administered together with L-DOPA. Inhibition of peripheral decarboxylation and O-methylation of L-DOPA allows more L-DOPA entry into the brain from relatively smaller doses of L-DOPA i.e. the conversion rate of ingested L-DOPA to useful dopamine within the brain is markedly enhanced. It has been known to treat Parkinson's disease with combination therapy utilizing L-DOPA and carbidopa (Sinemet™ and Sinemet-CR™ of Merck, Inc., New Jersey), or L-DOPA, carbidopa, and entacapone (found in Stalevo™ of Novartis Pharmaceutical, Inc., of Geneva, Switzerland). However, while the use of Stalevo was claimed to provide some advantage over Sinemet, present treatment protocol continues to suffer from some unwanted effects due to the pharmacodynamics of the dose and time courses of the drug.

### SUMMARY OF THE INVENTION

In one embodiment of the present invention, a composition comprising D-DOPA and a COMT inhibitor is provided.

In another embodiment of the present invention, a composition comprising D-DOPA and a COMT inhibitor, and optionally L-DOPA and optionally a peripheral amino acid decarboxylase inhibitor is provided.

In one embodiment of the present invention, a composition comprising a D-DOPA, L-DOPA, a peripheral amino acid decarboxylase and a COMT inhibitor is provided.

In a further embodiment of the present invention, a method of treating Parkinson's disease includes administering a therapeutically effective amount of D-DOPA and a COMT inhibitor is provided. The method may further include administering a therapeutically effective amount of L-DOPA and/or a peripheral amino acid decarboxylase inhibitor.

In another embodiment, a method of increasing the bioavailability of dopamine in the central nervous system, preferably the brain, comprising administering D-DOPA and a COMT inhibitor is provided. The method may further include administering L-DOPA and/or a peripheral decarboxylase inhibitor.

### BRIEF DESCRIPTION OF THE DRAWINGS

The present invention is illustrated by way of example, and not limitation, in the figures of the accompanying drawings in which like references denote similar elements, and in which:

FIG. 1 illustrates a dopamine pathway.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

The methods as described herein for use contemplate prophylactic use as well as curative use in therapy of an existing condition. As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with a D-DOPA, can include, but is not limited to, providing D-DOPA systemically to a patient by, e.g., intravenous injection whereby the therapeutic reaches the target tissue; oral ingestion, whereby the therapeutic reaches the target tissue. Administering a composition may be accomplished by injection, topical or oral administration, or by any method in combination with other known techniques.

As used herein “prodrug” denotes a derivative of a known direct acting drug, which derivative has enhanced delivery characteristics and therapeutic value as compared to the drug, and is transformed into the active drug by an enzymatic or chemical process.

As used herein, the term “therapeutic” means an agent utilized to treat, combat, ameliorate, prevent or improve an unwanted condition or disease of a patient. In part, embodiments of the present invention are directed to treat or prevent Parkinson's disease or ameliorate the symptoms of Parkinson's disease.

A “therapeutically effective amount” or “effective amount” of a composition is a predetermined amount calculated to achieve the desired effect, i.e., to treat or prevent Parkinson's disease or the symptoms associated with Parkinson's disease. A therapeutically effective amount of D-DOPA of the present invention is typically an amount such that when it is administered in a physiologically tolerable excipient composition, it is sufficient to achieve an effective systemic or local concentration in the tissue. Effective amounts of compounds of the present invention can be measured by improvements in patient symptoms and the like.

One embodiment of the present invention is a composition comprising D-DOPA and a COMT inhibitor. The COMT inhibitor may be selected from the group consisting of entacapone (Comtan®), tolcapone (Tasmar®) RO41-0960, OR-486, BIA 3-202 or dinitrocatechol (DNC). The composition may contain a therapeutically effective amount of D-DOPA and a therapeutically effective amount of a COMT inhibitor. In preferred embodiments, the composition comprises D-DOPA and entacapone or tolcapone.

COMT inhibitors are useful adjuncts to L-DOPA in the treatment of Parkinson's disease as they provide increased availability of the amino acid. Certain COMT inhibitors, including, for example DNC, penetrate the blood-brain barrier whereas others, including for example, entacapone, are restricted to extra-cerebral inhibition of COMT. It has been reported that entacapone administered at 30 mg/kg produced the same degree of liver and brain COMT inhibition as DNC. Brannan, T. et al., J. Neural. Transm. 1997; 104(a)₇₇-87. However at 2.5 and 5 mg/kg, entacapone achieved differential inhibition of liver and brain COMT activity of 80% and 10-30% respectively. BIA 3-202 (1-[3,4-dihydroxy-5-nitrophenyl]-2-phenyl-ethanone) is a new long-acting COMT inhibitor with limited access to the brain.

D-DOPA is readily available from Aldrich (Milwaukee, Wis.). D-DOPA can also be isolated from a racemic mixture of DOPA according to the procedures set forth in U.S. Pat. No. 3,405,159. A further method for the chemical preparation of D-DOPA is set forth in Yamada et al., Chem. Pharm. Bull., Vol. 10, No. 8, 693 (1962). The pharmaceutically acceptable salts can be prepared by reacting D-DOPA with an acid such as hydrochloric acid.

In another embodiment of the present invention, a composition comprising D-DOPA and a COMT inhibitor, and optionally L-DOPA and optionally a peripheral amino acid decarboxylase inhibitor is provided. Peripheral amino acid decarboxylase inhibitors may include, for example, carbidopa and benserazide. Preferred embodiments include the peripheral amino acid decarboxylase inhibitor carbidopa. The L-DOPA of such a composition may comprise an ester of levodopa, including, but not limited to a methyl-ester of levodopa (LDME or melevodopa) or ethyl ester of levodopa.

In one embodiment of the present invention, a composition comprising a D-DOPA, L-DOPA, a peripheral amino acid decarboxylase and a COMT inhibitor is provided. In a preferred embodiment, the composition comprises a racemic mixture of D-DOPA, L-DOPA, a COMT inhibitor and a peripheral amino acid decarboxylase inhibitor.

In another embodiment of the present invention, a method of treating Parkinson's disease includes administering a therapeutically effective amount of D-DOPA and a COMT inhibitor is provided. The method may further include administering a therapeutically effective amount of L-DOPA and/or a peripheral amino acid decarboxylase inhibitor. The L-DOPA of such a composition may comprise an ester of levodopa, including, but not limited to a methyl-ester of levodopa (LDME or melevodopa) or ethyl ester of levodopa.

In such methods of treating Parkinson's disease, the compounds may be administered together or separate and may be administered on the same or staggered dosing regimens. Given the observed increase in time to achieve maximum conctrentation of dopamine in the central nervous system following intragastric admiministratin of D-DOPA as compared to L-DOPA (2 hours and 1 hour, reseptively), a therapeutic regimen that utilizes these differences may be created. For example, a therapy may include a first administration of D-DOPA and a COMT inhibitor and L-DOPA. L-DOPA would be responsible for the initial dopamine levels in the CNS and then as the dopamine level from L-DOPA begins to decrease, dopamine formed from D-DOPA would begin to increase, providing an overall even level of dopamine in the CNS. Given the pharmacokinetic properties of COMT inhibitors and optionally peripheral amino acid decarboxylase inhibitors, subsequent doses of L-DOPA alone could be administered to maintain the level of dopamine in the CNS.

In another embodiment, a method of increasing the bioavailability of dopamine in the central nervous system, preferably the brain, comprising administering D-DOPA and a COMT inhibitor is provided. The method may further include administering L-DOPA and/or a peripheral decarboxylase inhibitor. In a preferred embodiment, the peripheral amino acid decarboxylase inhibitor may be carbidopa. In another preferred embodiment, the COMT inhibitor may be entacapone (Comtan®), tolcapone (Tasmar®) RO41-0960, OR-486, BIA 3-202 or dinitrocatechol (DNC).

In a further embodiment, a method of decreasing dopamine formation peripherally comprises administration of D-DOPA and a COMT inhibitor and optionally, L-DOPA and a peripheral amino acid decarboxylase inhibitor is provided.

It has been previously reported that D-DOPA is not a substrate for L-amino acid decarboxylase, yet D-DOPA can induce circling in rats with unilateral 6_hydroxydopamine lesion of the substantia nigra (M. J. Siddique and P. B. Silverman, 218, Neurosci. Lett. 145-148, (1996). However, it has since been reported that in the intact rat, the intragastric administration of D-DOPA together with carbidopa, a peripheral amino acid decarboxylase inhibitor, increased striatal dopamine concentration to the same extent as a similar treatment with L-DOPA plus carbidopa. Karoum F., et al., 440 Brain Research 190-194 (1988). This effect was attributed to conversion of D-DOPA to L-DOPA via D-amino acid oxidase and transamination of D-DOPA to dihydroxyphenylpyruvic acid then back to L-DOPA.

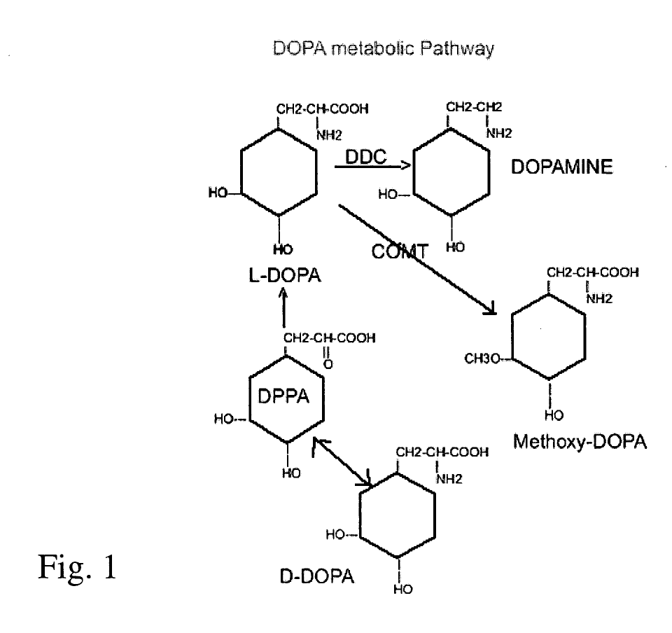

FIG. 1 illustrates a DOPA pathway which supports one or more embodiments of the present invention. The DOPA pathway of FIG. 1 is representative of pathways that produces dopamine from D-DOPA and L-DOPA. Results of previous studies have indicated that D-DOPA is converted to dopamine via transamination and/or D-amino acid oxidation to 3,4-dihydroxy-phenylpyruvic acid, which upon further transamination, gives rise to L-DOPA and hence dopamine. The benefits of L-DOPA in the treatment of Parkinson's disease are believed to result from its ability to increase striatal dopamine content via decarboxylation of L-DOPA by L-amino acid decarboxylase. Because this enzyme is stereo-specific, D-DOPA would not be expected to generate dopamine as efficient as L-DOPA. Therefore, administration of equal amounts of D/L DOPA and L-DOPA would not be expected to increase rat brain deuterated dopamine content to a similar extent as observed.

While not wishing to be bound by theory, we expect the administration of such peripheral COMT inhibitors, including, for example entacapone, tolcapone, BIA 3-202 (1-[3,4-Dihydroxy-5-nitrophenyl]-2-phenyl-ethanone) or dinitrocatechol (DNC) to produce two therapeutically important effects: protection of the administered D-DOPA from O-methylation, as well as protection of any L-DOPA formed peripherally (from D-DOPA by the above mentioned metabolic pathways) from O-methylation and by extension, maximizing and minimizing, respectively, the amount of D-DOPA or L-DOPA reaching the brain and the effective required dose.

As described herein, a method for treating Parkinson's disease includes administering to a patient suffering from this disease an anti-parkinsonism effective amount of the racemic form of DOPA (D,L-DOPA) in combination with effective amounts of a peripheral amino acid decarboxylase inhibitor and a COMT inhibitor. In an alternative embodiment, the method includes co-administering a pharmaceutically acceptable carrier or diluents. In an alternative embodiment, the D,L-DOPA is administered in an amount of 50 mg/kg body weight. In an alternative embodiment, the D,L-DOPA is administered in an amount of 50 to 500 mg per day. In an alternative embodiment, the D,L-DOPA is administered in an amount of peripheral amino acid decarboxylase inhibitor is carbidopa. In an alternative embodiment, the method includes administering an anti-cholinergic drug in an anti-parkinsonism effective amount. In an alternative embodiment, the method includes administering a substance in an anti-parkinsonism effective amount to alleviate a symptom selected from the group consisting of dyskinesia, dystonia, dysarthia, and dysphagia. In an alternative embodiment, the method includes administering to a patient suffering from this disease an anti-parkinsonism effective amount of D-DOPA in combination with effective amounts of a peripheral amino acid decarboxylase inhibitor and a COMT inhibitor.

One of ordinary skill in the art would appreciate that any of the substances mentioned in any of the embodiments described herein may be administered individually in a separate delivery mechanism (e.g., including but not limited to pills, capsules, tablets, liquid), or in combination in one or more pills, or through injection.

The compounds utilized in the present invention may be formulated into pharmaceutical compositions by combination with appropriate pharmaceutically acceptable carriers or diluents, and may be formulated into preparations in solid, semisolid, or liquid forms such as tablets, capsules, powders, granules, solutions, suppositories, or injections, in the usual ways for oral or parenteral administration. The following methods and experiments are merely exemplary and are in no way limiting. In pharmaceutical dosage forms, the compounds employed in the present invention may be used in the form of their pharmaceutically acceptable salts, and also may be used alone or in appropriate association, as well as in combination with other pharmaceutically active compounds such as carbidopa and other peripheral amino acid decarboxylase and COMT inhibitors. In the case of oral preparations, the compounds may be used alone or combined with appropriate additives to make tablets, powders, granules or capsules, e.g., with conventional additives such as lactose, mannitol, corn starch or potato starch; with binders, such as crystalline cellulose, cellulose derivatives, acacia, corn starch or gelatins; with disintegrators such as corn starch, potato starch or sodium carboxymethylcellulose; with lubricants such as talc or magnesium stearate; and if desired, with diluents, buffering agents, moistening agents, preservatives and flavoring agents. Furthermore, they may be made into suppositories by mixing with a variety of bases such as emulsifying bases or water-soluble bases. The compounds used in the present invention may be formulated into preparations for injections by dissolving, suspending or emulsifying them in aqueous or non-aqueous solvents, such as vegetable oil, synthetic aliphatic acid glycerides, esters of higher aliphatic acids or propylene glycol; and if desired, with conventional additives such a solubilizers, isotonic agents, suspending agents, emulsifying agents, stabilizers and preservatives.

A preparation made in accordance with an alternative embodiment of the present invention includes an anti-cholinergic drug in an anti-parkinsonism effective amount. Alternatively, a preparation made in accordance with an alternative embodiment of the present invention includes a substance in an anti-parkinsonism effective amount to alleviate at least one symptom including, but not limited to, dyskinesia, dytnonia, dysarthia, and dysphagia.

The suitable dose of D,L-DOPA varies with the subject, drug form, method and period of administration. However, in order to obtain desirable effects, generally it is recommended to administer the D,L-DOPA in amounts of 10-50 mg/kg body weight. More particularly, it is recommended to administer 100-500 mg D,L-DOPA per day. Instead of D,L-DOPA being administered as just described, D-DOPA alone may be alternatively administered.

The compositions of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compositions of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of the composition to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

For example, another embodiment of the present invention provides a composition of a D-DOPA of the present invention suitable for the treatment or prevention of Parkinson's disease by administering such a composition. Compositions suitable for treating diseases include, but are not limited to, pastes, gels, gums, topical liquids, sprays, inhalants or implantable devices for release into the oral tissue.

Pharmaceutical formulations of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present invention can be formulated for parenteral administration by injection, e.g., by bolus injection or continuous infusion. D-DOPA can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the D-DOPA compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The D-DOPA compositions of the present invention can also be formulated in rectal compositions such as suppositories or retention enemas, e.g., containing conventional suppository bases such as cocoa butter or other glycerides.

In addition to the formulations described previously, D-DOPA of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the D-DOPA compositions of the present invention, for example, can be applied to a plaster, or can be applied by transdermal, therapeutic systems that are consequently supplied to the organism.

Pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present invention can also be administered in combination with other active ingredients, such as, for example, adjuvants, or other compatible drugs or compounds where such combination is seen to be desirable or advantageous in achieving the desired effects of the methods described herein (e.g., controlling symptoms of Parkinson's disease). For example, D-DOPA compositions of the present invention can be administered with other inhibitors, including but not limited to, carbidopa or COMT inhibitors.

The following examples will serve to further typify the nature of this invention but should not be construed as a limitation in the scope thereof, which scope is defined solely by the appended claims.

### EXAMPLE 1

The present example illustrates the efficacy of the administration of D-DOPA of L-DOPA in vivo. In order to show the effectiveness of D-DOPA for increasing striatal dopamine content, male Sprague-Dawley Rats (Zivic-Miller, Allison Park, Pa.) weighing 300 to 400 grams were used. At least four weeks prior to the experiments, unilateral Substantia Nigra lesions were produced with intranigral administration of 6-hydroxydopamine according to the procedures set forth in Ungerstedt, Acta Physiol. Scand. (Suppl.) 367, 69 (1971).

The extent of unilateral Substantia Nigra lesions was tested by measuring apomorphine simulated rotation according to the procedures described in Understedt, supra, 1971; Freed et al. Ann. Neurol. 8, 510 (1980).

Either D- or L-DOPA was intragastrically administered to the rats in combination with carbidopa at doses of 50 mg/kg body weight of D-DOPA or L-DOPA and 5 mg/kg body weight of carbidopa, suspended in sterile water, to provide a pharmaceutically acceptable solution for administration. D- or L-DOPA was intragastrically administered to groups of five rats, which were sacrificed one or two hours after treatment. The left (intact) and right (lesioned) striata of these rats were excised and analyzed separately for the content of dopamine and its metabolites 3,4-dihydroxyphenylacetic acid (DOPAC) and homovanillic acid (HVA), according to the procedure as set forth in Karoum, Neuromethods, Vol. 2 (G. B. Baker, A. A. Boulton and J. M. Baker eds.), The Humana Press, Inc., New Jersey, page 305, 1985 (referred to hereafter as “Karoum, Neuromethods”).

TABLE 1
The effects of (L-DOPA + carbidopa) and (D-DOPA + carbidopa) on striatal
concentration of dopamine, DOPAC and HVA in rats with unilateral substantia nigra lesions
All results arc in (mean + S.E.M.) ng/mg protein.

| | Dopamine | | DOPAC | | HVA | |
|---|---|---|---|---|---|---|
| | Intact | Lesioned | Intact | Lesioned | Intact | Lesioned |
| Description | striatum | striatum | striatum | striatum | striatum | striatum |
| Unilaterally lesioned rats | 150 ± 8.3 | 4.5 ± 1.4 | 17 ± 0.9 | 0.8 ± 0.3 | 6.9 ± 1.5 | 1.0 ± 0.3 |
| ULR 1 h after L-DOPA plus | 180 ± 5.1* | 2.0 ± 0.2 | 38 ± 4.8* | 12 ± 2.6** | 26 ± 3.3** | 11 ± 1.8** |
| carbidopa″ | | | | | | |
| ULR 1 h after D-DOPA plus | 170 ± 11.2 | 3.4 ± 0.7 | 46 + 4.8″ | 17 + 2.2** | 27 ± 2.3** | 11 ± 1.8** |
| carbidopa^{b} | | | | | | |
| ULR 2 h after L-DOPA plus | 160 ± 5.8 | 2.5 ± 1.0 | 22 ± 2.2* | 5.6 ± 1.4** | 13 ± 1.2** | 4.7 + 0.9** |
| carbidopa″ | | | | | | |
| ULR 2 h after D-DOPA plus | 190 ± 4.2* | 2.8 + 1.1 | 18 ± 1.6 | 4.5 ± 1.1** | 10 ± 0.9 | 4.3 ± 0.7** |
| carbidopa¹¹ | | | | | | |

^{a} L-DOPA consisted of 50 mg/kg of L-DOPA plus 5 mg/kg of carbidopa.
^{b}D-DOPA consisted of 50 mg/kg of D-DOPA plus 5 mg/kg of carbidopa. L- and D-DOPA were administered intragastrically in a water suspension. Rats in groups of 5 were sacrificed 1 or 2 h after treatment.
*P < 0.05, compared with untreated unilaterally lesioned rats (unpaired t-test);
**P < 0.005, compared with untreated unilaterally lesioned rats (unpaired Mest).

As can be seen from the results in Table 1, intragastric administration of D- and L-DOPA increased the concentrations of dopamine and its metabolites in the intact striata of the unilaterally lesion rats to about the same extent, but there appeared to be a delay in the time required for D-DOPA to produce its maximal effect. Dopamine content in the intact striata peaked at one hour after L-DOPA administration and two hours after D-DOPA. Neither D- nor L-DOPA increased dopamine concentrations in the lesion striata. Rather, dopamine concentrations were greatly reduced as a result of the unilateral lesion of the substantia nigra. The concentrations of DOPAC and HVA in the lesion striata were markedly increased by both D- and L-DOPA one hour after treatment and then rapidly declined two hours thereafter, indicating that the formation of dopamine from D- and L-DOPA in the striatum reached its maximum concentration within one hour after intragastric administration.

### EXAMPLE 2

The present example illustrates the possible involvement of DHPPA in the formation of dopamine from D-DOPA. Since L-amino acid decarboxylase is stereospecific, direct decarboxylation by the enzyme does not appear to account for the substantial formation of dopamine from D-DOPA. In an attempt to examine an alternate pathway, four groups of Sprague-Dawley rats weighing 120-150 grams with cannulae permanently implanted into their lateral ventricles, according to the procedure described in Robinson et al., Physiol. Behav. 4, 123-124, (1969), were used. One group received 10 μl. of saline into the ventricles. The second, third and fourth groups received, intraventricularly, 200.μ.g of L-DOPA, D-DOPA and 3,4-dihydroxyphenylpyruvic acid (DHPPA) in 10 μl of saline, respectively. The rats were sacrificed two hours after treatment and their striata removed and analyzed. Striatal concentrations of dopamine, DOPAC and HVA were measured by massfragmentography according to the procedure set forth in Karoum, Neuromethods.

TABLE 2
The effects of intraventricular administration of D-DOPA,
L-DOPA and 3.4-dihydroxyphmylpynwic add (DHPPA) on
straital dopmnie and its metabolites (DOPAC and HVA)

| Description | Dopamine | DOPAC | HVA |
|---|---|---|---|
| Control fits | 99 ± 1.4 | 16 ± 0.43 | 5.2 ± 0.23 |
| Intraventricular | 120 ± 7.4* | 59 ± 9.49* | 22.2 ± 1.60* |
| L-DOPA^{a} | | | |
| Intraventricular | 116 ± 5.1* | 19 ± 0.92 | 4.8 ± 0.50 |
| D-DOPA^{a} | | | |
| Intraventricular | 140 ± 11.0* | 96 ± 16.6* | 23.3 ± 3.53* |
| DHPPA^{a} | | | |

All results are in (mean ± S.E.M.) ng/mg protein.
^{a}200 as of each of L-DOPA.
D-DOPA or DHPPA in volumes of 10 μl, was injected intraventricularly via permanently cannulae in the ventricles. The rats were sacrificed 2 h after treatment and their brains removed.
*P < 0.05 compared to the controls by analysis of variance employing the Bonferroni correction.

As seen from Table 2, all three treatments significantly increased striatal concentrations of dopamine and its metabolites. The increases in DOPAC and HVA produced were higher for L-DOPA and 3,4-dihydroxyphenylpyruvic acid (DHPPA) than for D-DOPA. The results indicate that DHPPA is readily converted to dopamine in the brain. In fact, intraventricular administration of DHPPA produced larger elevations of dopamine and DOPAC than did either D- or L-DOPA. On the other hand, repeated intragastric administration of DHPPA plus carbidopa (50 mg/kg of each for four days) did not increase striatal dopamine or its metabolites, confirming that DHPPA does not easily cross the blood brain barrier. Hence, if DHPPA is an intermediate in the conversion of D-DOPA to dopamine, the metabolic changes probably occurred within the brain. The accumulation of dopamine in the striatum of rats receiving DHPPA intraventricularly, taken together with the wide distribution of the transamination reaction in both the brain and peripheral tissues, favorably supports the possible involvement of DHPPA in the formation of dopamine from D-DOPA.

DHPPA can be formed from D-DOPA by either of two pathways, that is, direct transamination, or through deamination by D-amino acid oxidase, an enzyme widely distributed in the brain. The pattern of changes of striatal dopamine and its metabolites observed after intragastric administration of D- and L-DOPA are similar to those observed in the hypothalamus. Both amino acids increase hypothalamic dopamine by similar amounts, while the increases in DOPAC and HVA are considerably higher after administration of L-DOPA than after the administration of D-DOPA.

### EXAMPLE 3

The present example illustrates the effects of carbidopa on the urinary excretion of dopamine after the administration of D- and L-DOPA alone, (50 mg/kg). Carbidopa reduced the excretion of dopamine following D-DOPA to a far greater degree than when carbidopa was co-administered with L-DOPA as set forth in Karoum et al., Brain Research, 1988. These results suggest that the pathways responsible for the formation of dopamine from D-DOPA in the periphery are more sensitive to carbidopa than are the pathways that convert L-DOPA to dopamine. Hence, one might expect that in rats receiving equal doses of either stereoisomer, proportionately more D-DOPA than L-DOPA will cross into the brain when each of these amino acids is co-administered with carbidopa. The unexpected ability of D-DOPA to elevate striatal concentrations of dopamine suggests that D-DOPA offers advantages over L-DOPA in the treatment of Parkinson's disease. For example, the peripheral undesirable side effects normally associated with L-DOPA treatment e.g., nausea, vomiting, cardiac arrthymias, hypotension and diarrhea could be lessened by the use of D-DOPA. Furthermore, since in the brain the conversion rate of D-DOPA to dopamine is slower than that of L-DOPA, a more adequate dosing system can be achieved and, hence, a better steady state concentration of striatal dopamine can be obtained.

### EXAMPLE 4

The following example illustrate pharmaceutical preparations which can be made and utilized in the present invention.

1000 g of D-DOPA, 250 g of carbidopa, and 2000 g of entacapone, 750 g of microcrystalline cellulose (Avicel-PH-101), 50 g of stearic acid, 100 g of colloidal silica are granulated and blended. Tablets are punched using a 7/16 inch standard concave punch to obtain 10,000 tablets each containing 100 mg of D-DOPA, 200 mg entacapone, and 25 mg of carbidopa.

The foregoing is an illustrative example, and one of ordinary skill in the art would appreciate that preparations with lower or greater strengths may also be prepared. The foregoing formulation may be scaled up or down by 75%. As indicated elsewhere herein, D,L-DOPA may be used instead of D-DOPA.

In the preceding specification, the invention has been described with reference to specific exemplary embodiments of the invention. It will, however, be evident to one of ordinary skill in the art that various modifications and changes may be made without departing from the broader spirit and scope of the invention as set forth in the claims that follow. The specification and drawings are accordingly to be regarded in an illustrative rather than restrictive sense.

What is claimed is:

1. A pharmaceutical composition comprising an effective amount of D-DOPA and a COMT inhibitor.

2. The composition of claim 1, further comprising a therapeutically effective amount of L-DOPA.

3. The composition of claim 2, wherein said COMT inhibitor is entacapone.

4. The composition of claim 3, wherein said entacapone is in a peripherally effective amount.

5. The composition of claim 3, wherein said entacapone is in a centrally effective amount.

6. The composition of claim 2, wherein said COMT inhibitor is selected from the group consisting of tolcapone, RP41-0960, OR-486, and BIA 3-202.

7. The composition of claim 2, wherein said COMT inhibitor is dinitrocatechol.

8. The composition of claim 2, wherein said L-DOPA and D-DOPA are at substantially similar quantities.

9. The composition of any one of claim 1, further comprising a decarboxylase inhibitor.

10. The composition of claim 9, wherein said decarboxylase inhibitor is carbidopa.

11. The composition of claim 2, wherein said effective amount of D-DOPA is about 50-500 milligrams.

12. A method of treating Parkinson's disease comprising administering to a patient a therapeutically effective amount of D-DOPA and a COMT inhibitor.

13. The method of claim 12, further comprising administering a therapeutically effective amount of L-DOPA.

14. The method of claim 13, wherein said COMT inhibitor is entacapone.

15. The method of claim 14, wherein said entacapone is in a peripherally effective amount.

16. The method of claim 14, wherein said entacapone is in a centrally effective amount.

17. The method of claim 13, wherein said COMT inhibitor is selected from the group consisting of tolcapone, RP41-0960, OR-486, and BIA 3-202.

18. The method of claim 13, wherein said COMT inhibitor is dinitrocatechol.

19. The method of claim 13, wherein said L-DOPA and D-DOPA are administered at substantially similar quantities.

20. The method of claim 13, further comprising the administration of a decarboxylase inhibitor.

21. The method of claim 20, wherein said decarboxylase inhibitor is carbidopa.

22. The method of claim 12, wherein said therapeutically effective amount of D-DOPA administered is about 25 to about 150 mg/kg of said patient.

23. The method of claim 12 further comprising administering to said patient an effective amount of L-DOPA.

24. The method of claim 12 further comprising administering to said patient a decarboxylase inhibitor.

25. The method of claim 25, wherein said decarboxylase inhibitor is carbidopa.

26. A pharmaceutical composition of a centrally effective combination of D-DOPA and a COMT inhibitor.

27. The pharmaceutical composition of claim 26, wherein said combination consists of essentially of D-DOPA and a COMT inhibitor.

28. The pharmaceutical composition of claim 27, wherein said composition further comprises carbidopa.

29. The pharmaceutical composition of claim 28, wherein said COMT inhibitor is a centrally effective amount of entacapone.

30. The pharmaceutical composition of claim 28, wherein said COMT inhibitor is dinitrocatechol.
